(19) United States
(12) Patent Application Publication
(10) Pub. No.: US 2013/0184605 A1
(43) Pub. Date: Jul. 18, 2013

(54) SYSTEM AND DATA PROCESSING DEVICE FOR BIOIMPEDANCE ANALYSIS

(71) Applicants: Tak-shing Ching, Taichung City (TW); Tai-Ping Sun, Taoyuan County (TW); Wei-Hao Liu, Nantou (TW)

(72) Inventors: Tak-shing Ching, Taichung City (TW); Tai-Ping Sun, Taoyuan County (TW); Wei-Hao Liu, Nantou (TW)

(21) Appl. No.: 13/468,892

(22) Filed: May 10, 2012

(30) Foreign Application Priority Data
Jan. 13, 2012 (TW) 101101449

(51) Int. Cl. A61B 5/053

(52) U.S. Cl. 600/547

(57) ABSTRACT

A system for bioimpedance analysis of a biological tissue includes a tissue inspector and an impedance calculator. The tissue inspector includes an inspecting circuit for receiving a test signal, a plurality of electrode sets respectively corresponding with a plurality of test parts, and a multiplexer operable to couple a selected electrode set at a detected test part to the inspecting circuit to detect a signal response resulting from the test signal. The impedance calculator is operable to compute impedance information corresponding to the detected test part based on the test signal and the detected signal response.

### CROSS-REFERENCE TO RELATED APPLICATION

This application claims priority to Taiwanese Application No. 101101449, filed on Jan. 13, 2012.

### BACKGROUND OF THE INVENTION

1. Field of the Invention

The invention relates to a system and data processing device for bioimpedance analysis.

2. Description of the Related Art

Bioimpedance technology is used to analyze tissue structure by measuring impedance parameter of a biological tissue, thereby making it possible to determine whether the tissue has undergone physical change, such as oral pathological change or change due to skin cancer.

Typically, an impedance analyzer WK6420C, which is available from Wayne Kerr Electronics Inc., is used in combination with a current limiter to apply a constant voltage on the biological tissue and obtain an impedance value from an output current obtained from the tissue. The impedance analyzer is often used as a measurement tool clinically because of advantages, such as non-invasiveness and continuous monitoring. However, the aforesaid impedance analyzer can only provide impedance of a single test part in the biological tissue, such that the measurement result may not be an objective result.

### SUMMARY OF THE INVENTION

Therefore, an object of the present invention is to provide a system for bioimpedance analysis that can provide impedance information of a plurality of test parts of a biological tissue in a short amount of time.

According to one aspect of the present invention, a system for bioimpedance analysis of a biological tissue comprises:
a tissue inspector including:
   an inspecting circuit for receiving a test signal;
   a plurality of electrode sets disposed to respectively correspond with a plurality of test parts of the biological tissue; and
   a multiplexer operable to couple a selected one of the electrode sets corresponding to a detected one of the test parts to the inspecting circuit, wherein a signal response at the detected one of the test parts and resulting from the test signal is detected using the selected one of the electrode sets; and

an impedance calculator for computing impedance information corresponding to the detected one of the test parts based on the test signal and the signal response detected using the selected one of the electrode sets.

Another object of the present invention is to provide a data processing device adapted to receive a plurality of digital response signals that respectively correspond to a plurality of test parts of a biological tissue.

According to another aspect of the present invention, a data processing device comprises:

an impedance calculator for computing impedance information of each of the test parts of the biological tissue based on the digital response signal corresponding thereto;

an image generator operable to generate an image signal according to the impedance information from the impedance calculator and coordinate signals corresponding to respective locations of the test parts on the biological tissue; and

a judging unit operable to determine whether or not the biological tissue is normal according to the impedance information and the coordinate signals that correspond to the test parts of the biological tissue.

### BRIEF DESCRIPTION OF THE DRAWINGS

Other features and advantages of the present invention will become apparent in the following detailed description of the preferred embodiment with reference to the accompanying drawings, of which:

FIG. 1 is a block diagram illustrating a preferred embodiment of the system for bioimpedance analysis according to the present invention;

FIG. 2 is a schematic circuit diagram showing a signal generator of the preferred embodiment;

FIG. 3 is a schematic circuit diagram showing a tissue inspector and a differential amplifier of the preferred embodiment;

FIG. 4 is a schematic diagram illustrating an image obtained for a normal tissue sample;

FIG. 5 is a schematic diagram illustrating an image obtained for a tissue that has undergone pathological change; and

FIGS. 6 and 7 are plots showing simulation results using the preferred embodiment and the impedance analyzer WK6420C.

### DETAILED DESCRIPTION OF THE PREFERRED EMBODIMENT

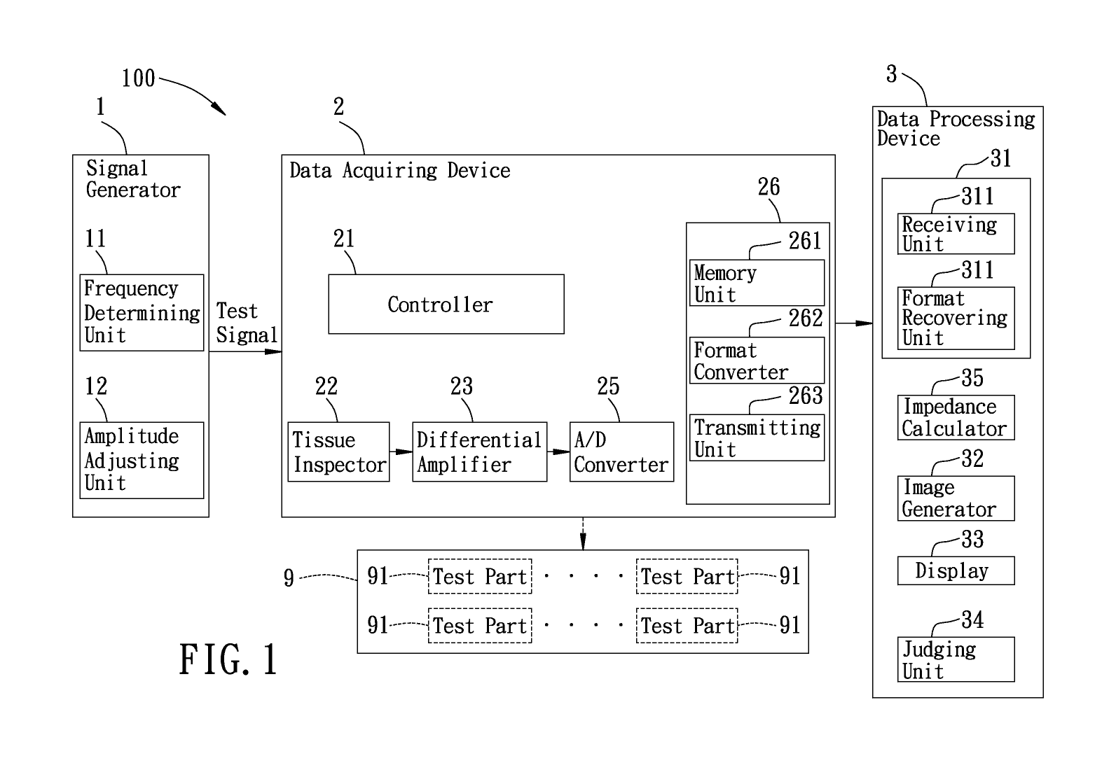

Referring to FIG. 1, the preferred embodiment of the system 100 for bioimpedance analysis according to this invention is adapted to analyze a biological tissue 9, and comprises a signal generator 1, a data acquiring device 2, and a data processing device 3. The signal generator 1 is operable to generate a test signal. The data acquiring device 2 is operable to apply the test signal to a plurality of test parts 91 of the biological tissue 9 so as to obtain a signal response from each test part 91. The data processing device 3 includes an impedance calculator 35 for computing impedance information of each of the test parts 91 based on the signal response corresponding thereto, an image generator 32 operable to generate an image signal according to the impedance information of the whole biological tissue 9, and a display 33 for displaying an image corresponding to the image signal.

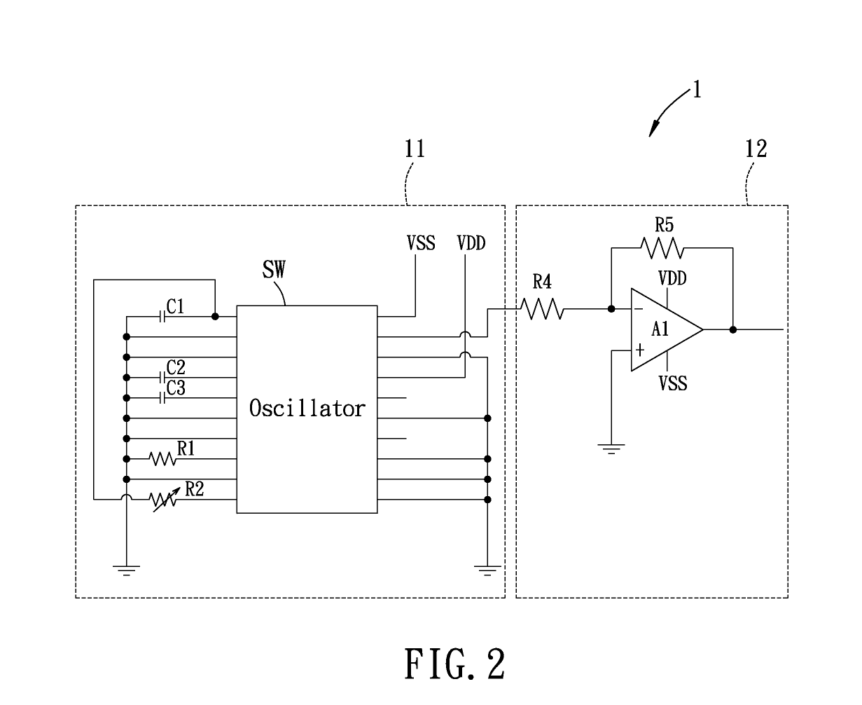

Referring to FIG. 2, the signal generator 1 includes a frequency determining unit 11 and an amplitude adjusting unit 12. The frequency determining unit 11 includes an oscillator SW operable to generate the test signal in a form of a sine wave with a frequency dependent upon resistors R1, R2 and capacitors C1, C2, and C3. The amplitude adjusting unit 12 is operable to adjust amplitude of the test signal based upon resistors R4 and R5. Amplitude of the test signal must be reduced using the amplitude adjusting unit 12 because the biological tissue 9 is not suited to receive a signal with a large amplitude. The test signal from the amplitude adjusting unit 12 is in a form of an alternating current sine-wave voltage. In this embodiment, the oscillator SW is implemented using a MAX038 chip available from Maxim Integrated Products Inc., and the amplitude adjusting unit 12 is implemented using an OPA37 component available from Texas Instruments Inc., but the invention should not be limited in this respect.

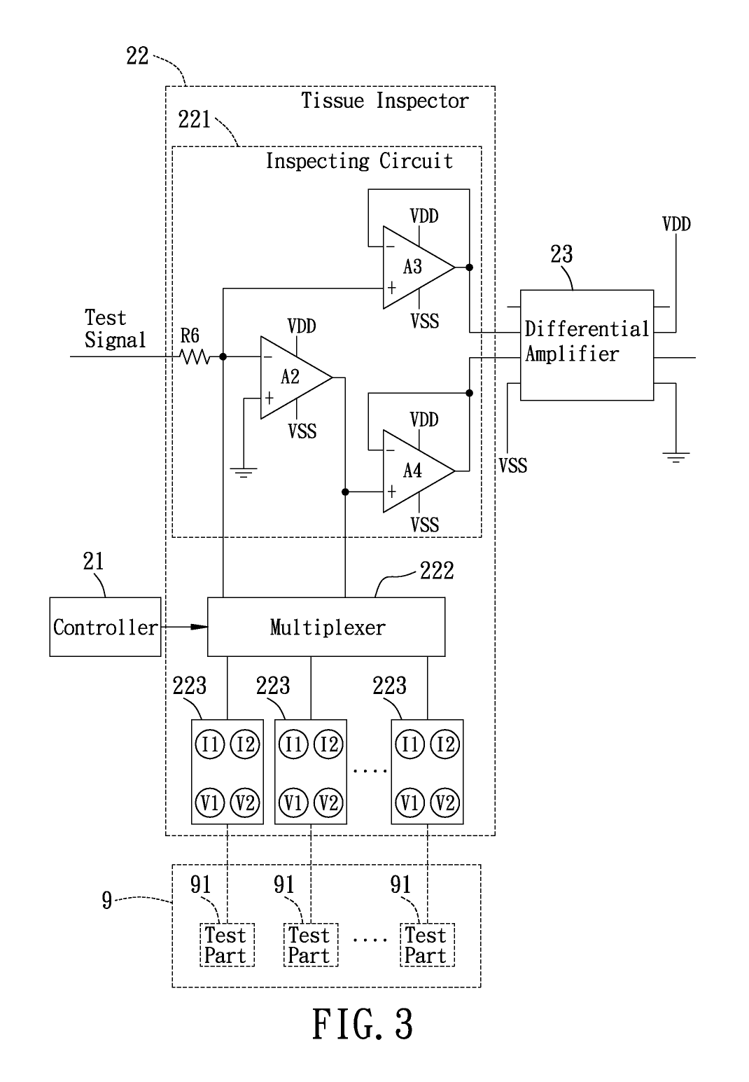

Referring to FIG. 1 and FIG. 3, the data acquiring device 2 includes a controller 21, and further includes a tissue inspector 22, a differential amplifier 23, and an analog-to-digital (A/D) converter 25 connected in series in the aforesaid sequence.

The tissue inspector 22 is operable to convert the test signal from a voltage form into a current form, and is controlled by the controller 21 to sequentially inspect the test parts 91 of the biological tissue 9 based on the converted current-form test signal. The differential amplifier 23 is operable to acquire the signal response from the corresponding test part 91. The A/D converter 25 is operable to convert the signal response acquired by the differential amplifier 23 from an analog response signal into a digital response signal.

In detail, the tissue inspector 22 includes an inspecting circuit 221 for receiving the test signal, a multiplexer 222 and a plurality of electrode sets 223 that are disposed adjacent to each other. The inspecting circuit 221 includes a resistor R6 and an amplifier A2. The resistor R6 is coupled between the amplitude adjusting unit 12 and an inverting input of the amplifier A2. Each electrode set 223 includes a first current electrode 11, a second current electrode 12, a first voltage electrode V1, and a second voltage electrode V2. Preferably, the electrode sets 223 are arranged in a matrix, and each corresponds to a coordinate. When the tissue inspector 22 is attached to the biological tissue 9, the electrode sets 223 are coupled to the test parts 91 of the biological tissue 9, respectively.

The multiplexer 222 is controlled by the controller 21 to sequentially couple the electrode sets 223 to the inspecting circuit 221 to inspect the test parts 91. When a selected electrode set 223 is coupled to the inspecting circuit 221, the first current electrode 11 and the first voltage electrode V1 thereof are coupled to the inverting input of the amplifier A2 through the multiplexer 222, and the second current electrode 12 and the second voltage electrode V2 thereof are coupled to an output of the amplifier A2 through the multiplexer 222. A non-inverting input of the amplifier A2 is grounded in this embodiment.

The inspecting circuit 221 converts the test signal from a voltage form to a current form through the resistor R6. In this embodiment, when the selected electrode set 223 is coupled to the inspecting circuit 221, the current-form test signal flows from the inspecting circuit 221 to the corresponding detected test part 91 through the resistor R6 and the first current electrode 11, and returns through the second current electrode 12, so as to obtain a voltage-form signal response through the first voltage electrode V1 and the second voltage electrode V2. The amplifier A2 in this embodiment is a UA741 component available from Texas Instrument Inc., which has a high input impedance characteristic that permits flow of almost the entire current-form test signal to the detected test part 91.

Moreover, in order to reduce possibility of current flowing to the differential amplifier 23, the inspecting circuit 221 further includes a first buffer A3 coupled between the inverting input of the amplifier A2 and a first input of the differential amplifier 23, and a second buffer A4 coupled between the output of the amplifier A2 and a second input of the differential amplifier 23. Accordingly, the differential amplifier 23 may acquire the signal response of the detected test part 91 more precisely. However, in other embodiments, the first and second buffers A3, A4 may be omitted. In this embodiment, the buffers A3, A4 are implemented using LM324 components available from STMicroelectronics Inc., and the multiplexer 222 is implemented using a CD4051 component available from Texas Instruments Inc. However, the invention should not be limited in this respect.

After the A/D converter 25 converts the signal response into the digital response signal, the impedance calculator 35 of the data processing device 3 is operable to compute the impedance information accordingly. Because the test signal is an alternating current signal, the digital response signal has a plurality of positive peak values and negative peak values. In this embodiment, the impedance information is computed by obtaining a direct current (DC) component of the digital response signal which is an average of the positive and negative peak values of the digital response signal, followed by dividing the DC component by a reference value related to the test signal.

When the controller 21 controls the multiplexer 222 such that the selected one of the electrode sets 223 corresponding to the detected one of the test parts 91 is coupled to the inspecting circuit 221, the controller 211 is operable to provide a two-dimensional coordinate signal corresponding to the coordinate of the selected electrode set 223, which corresponds to a location of the detected test part 91 on the biological tissue 9. Thereafter, the image generator 32 of the data processing device 3 is operable to generate the image signal according to the impedance information from the impedance calculator 35 and the coordinate signals corresponding to respective locations of the test parts 91 on the biological tissue, and the display 33 is operable for displaying the image corresponding to the image signal. Moreover, the data processing device 3 further includes a judging unit 34 operable to determine whether or not the biological tissue 9 is normal according to the impedance information and the coordinate signals that correspond to the test parts 91 of the biological tissue 9.

In this embodiment, after obtaining the signal response of one test part 91 through the corresponding electrode set 223, the multiplexer 222 is rapidly switched to couple another electrode set 223 to the inspecting circuit 22, such that inspection of all test parts 91 of the biological tissue 9 may be completed in a short amount of time. Compared to the conventional system, the preferred embodiment can provide an overall analysis of the biological tissue 9, not limited to a single test part, so as to obtain a result that is more objective.

In this embodiment, the signal generator 1 and the data acquiring device 2 are integrated in an electronic apparatus (not shown), and the data processing device 3 is implemented using a computer (not shown). Data transmission between the electronic apparatus and the computer may be accomplished through a wired RS-232 interface. Therefore, the data acquiring device 2 further includes a translator 26, and the data processing device 3 further includes an interpreter 31. The translator 26 has a memory unit 261 to store the coordinate signal and the digital response signal, a format converter 262 operable to perform an RS-232 format conversion on signals in the memory unit 261, and a transmitting unit 263 to transmit a format-converted signal. The interpreter 31 has a receiving unit 311 to receive the format-converted signal, and a format recovering unit 312 to perform a recovery operation to recover the coordinate signal and the digital response signal. Then, the coordinate signal and digital response signal thus recovered are provided to the impedance calculator 35 for computing the impedance information.

The image generator 32 is operable to represent the impedance information in R grayscale levels (in this embodiment, R=256). The image generator 32 is configured to obtain a maximum value H and a minimum value L among the impedance information for the test parts 91 of the biological tissue 9.

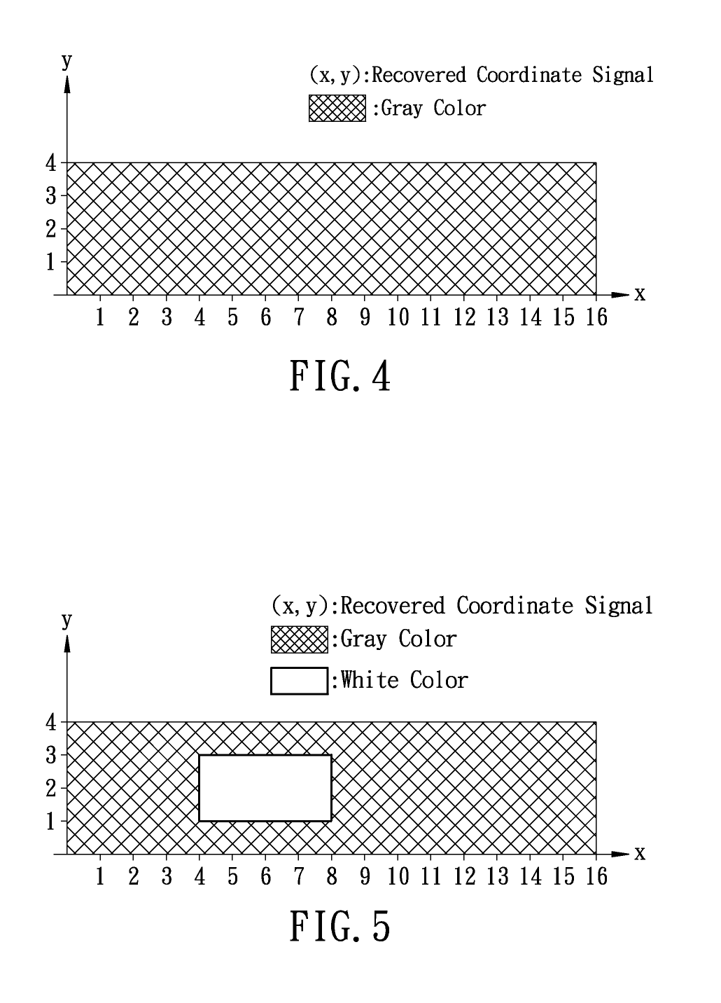

When a value M associated with a normal impedance range of the biological tissue 9 is closer to the maximum value H than the minimum value L, the image generator 32 is configured to generate grayscale values G according to G=D/[(H−M)/127], wherein D represents the impedance information, and 127 comes from 0.5R−1 (R=256). On the other hand, when the value M associated with the normal impedance range of the biological tissue 9 is closer to the minimum value L than the maximum value H, the image generator 32 is configured to generate the grayscale values G according to G=D/[(M−L)/127]. Preferably, the value M is an average of upper and lower limits of the normal impedance range of the biological tissue 9. Then, the image generator 32 is operable to generate a two-dimensional image signal according to the recovered coordinate signals and the corresponding grayscale values G, and the display 32 is operable to display the image corresponding to the image signal. Typically, impedance of a normal tissue ranges between 50000 and 80000 ohms, and that corresponds to a median gray color of the 256 grayscale color levels. Therefore, if the obtained image is that shown in FIG. 4, it can be inferred that impedances of the 16×4 test parts 91 are similar, and the grayscale values fall in the median portion of 256 grayscale color levels. The judging unit 34 determines the biological tissue 9 to be normal. On the other hand, if the obtained image is that shown in FIG. 5, the color corresponding to some of the test parts 91 is too white, and the judging unit 34 determines the biological tissue 9 to be one that may have undergone pathological changes.

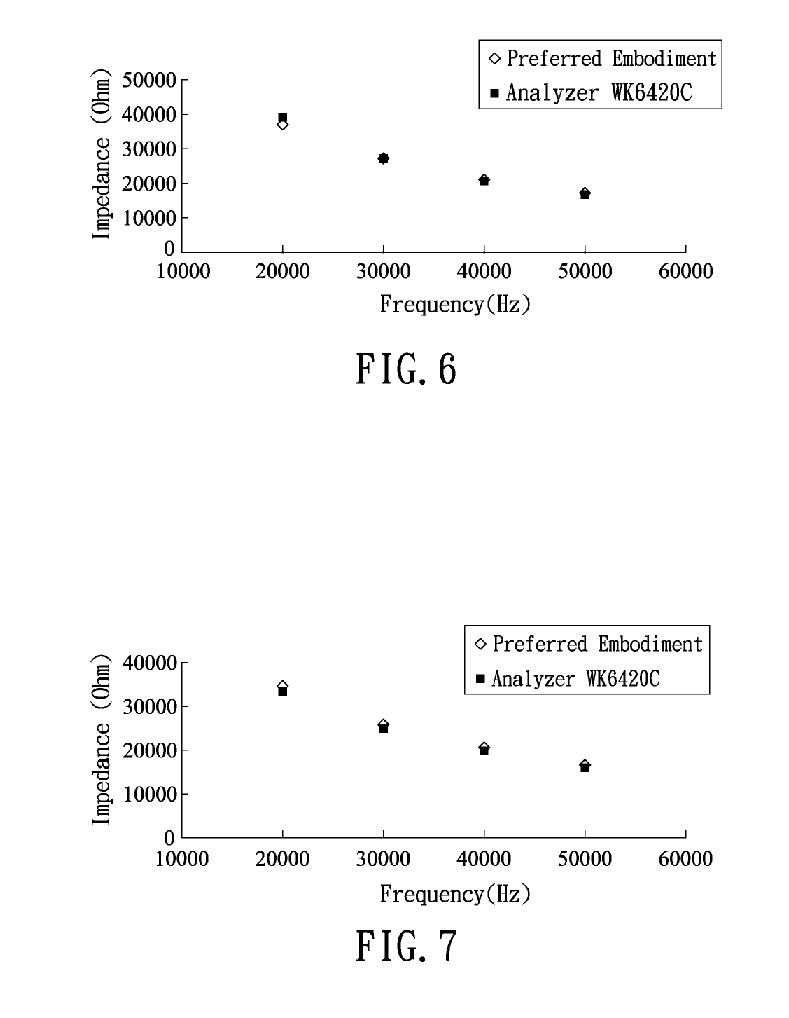

Compared to the A/D converter of the analyzer WK6420C, in order to reduce cost, the A/D converter 25 with lower resolution is selected in this embodiment. However, through a simulation experiment, even if test signals with different frequencies are applied, the obtained impedance information is very similar to that obtained using a WK6420C. This means that the obtained impedance information of the test parts 91 is not influenced by the lower resolution. FIG. 6 shows a result of applying test signals with different frequencies on a simulated test part 91 with 100000 ohms, while FIG. 7 shows a result of using a simulated test part 91 with 56000 ohms.

In other embodiments, the judging unit 34 may be omitted, and determination as to whether or not the biological tissue 9 has undergone pathological changes may be made by a user through direct observation of the image on the display 33.

It should be noted that, in the preferred embodiment, data transmission between the data acquiring device 2 and the data processing device 3 is through wired RS-232 interface. In other embodiments, another wired interface or wireless transmission may be used.

Moreover, the data acquiring device 2 and the data processing device 3 may be integrated into a single apparatus. Hence, data transmission does not need to go through the RS-232 interface. That is, the translator 26 and the interpreter 31 can be omitted, and the impedance calculator 35 can directly receive the digital response signal.

In addition, the first current electrode 11 and the first voltage electrode V1 can be coupled to the same node, and can be replaced with a first common electrode in other embodiments. Similarly, the second current electrode 12 and the second voltage electrode V2 can be replaced with a second common electrode.

To sum up, the data acquiring device 2 of this invention uses the multiplexer 222 to inspect a plurality of test parts 91 of the biological tissue 9 by switching, to thereby acquire a plurality of the impedance information of the test parts 91, and result in objective judgment of the tissue without incoming high costs.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A system for bioimpedance analysis of a biological tissue, said system comprising:
a tissue inspector including:
an inspecting circuit for receiving a test signal;
a plurality of electrode sets disposed to respectively correspond with a plurality of test parts of the biological tissue; and
a multiplexer operable to couple a selected one of said electrode sets corresponding to a detected one of the test parts to said inspecting circuit, wherein a signal response at the detected one of the test parts and resulting from the test signal is detected using the selected one of said electrode sets; and
an impedance calculator for computing impedance information corresponding to the detected one of the test parts based on the test signal and the signal response detected using the selected one of said electrode sets.

2. The system as claimed in claim 1, wherein each of said electrode sets includes a first electrode and a second electrode, said inspecting circuit including:
a resistor disposed to receive the test signal; and
an amplifier having an input coupled to said resistor, and an output;
wherein, when said multiplexer couples the selected one of said electrode sets to said inspecting circuit, said first electrode of the selected one of said electrode sets is coupled to said input of said amplifier through said multiplexer, said second electrode of the selected one of said electrode sets is coupled to said output of said amplifier through said multiplexer, the test signal is applied to the detected one of the test parts through said resistor and said first electrode, and the signal response includes a voltage between said first and second electrodes of the selected one of said electrode sets.

3. The system as claimed in claim 2, further comprising:
a differential amplifier having a first input coupled to said input of said amplifier of said inspecting circuit, and a second input coupled to said output of said amplifier of said inspecting circuit, said differential amplifier being operable to acquire the signal response of the detected one of the test parts according to signals at said first and second inputs thereof; and
an analog-to-digital converter operable to convert the signal response acquired by said differential amplifier into a digital response signal that is provided to said impedance calculator for computing the impedance information.

4. The system as claimed in claim 3, further comprising:
a first buffer coupled between said input of said amplifier and said first input of said differential amplifier; and
a second buffer coupled between said output of said amplifier and said second input of said differential amplifier.

5. The system as claimed in claim 1, further comprising a controller operable to control said multiplexer to couple the selected one of said electrode sets to said inspecting circuit, and operable to generate a coordinate signal corresponding to a location of the detected one of the test parts on the biological tissue.

6. The system as claimed in claim 5, further comprising an image generator operable to generate an image signal according to the impedance information and the coordinate signal that correspond to the detected one of the test parts.

7. The system as claimed in claim 6, wherein:
said image generator is configured to obtain a maximum value H and a minimum value L among the impedance information for the test parts of the biological tissue;
when a value M associated with a normal impedance range of the biological tissue is closer to the maximum value H than the minimum value L, said image generator is configured to generate grayscale values G according to G=D/[(H−M)/(0.5R−1)], wherein D represents the impedance information, and R represents a total number of grayscale levels; and
when the value M associated with the normal impedance range of the biological tissue is closer to the minimum value L than the maximum value H, said image generator is configured to generate the grayscale values G according to G=D/[(M−L)/(0.5R−1)].

8. The system as claimed in claim 7, wherein the value M associated with the normal impedance range of the biological tissue is an average of upper and lower limits of the normal impedance range of the biological tissue.

9. The system as claimed in claim 7, wherein said image generator is configured to generate the image signal according to the grayscale values G and the coordinate signals that correspond to the test parts of the biological tissue.

10. The system as claimed in claim 7, further comprising a judging unit operable to determine whether or not the biological tissue is normal according to the grayscale values and the coordinate signals that correspond to the test parts of the biological tissue.

11. The system as claimed in claim 1, wherein the signal response at the detected one of the test parts is an alternating current signal, and said impedance calculator is configured to compute the impedance information by obtaining a direct current (DC) component of the signal response, followed by dividing the DC component by a reference value related to the test signal.

12. The system as claimed in claim 11, wherein the signal response at the detected one of the test parts has a plurality of positive peak values and negative peak values, and the DC component is an average of the positive and negative peak values.

13. A data processing device adapted to receive a plurality of digital response signals that respectively correspond to a plurality of test parts of a biological tissue, said data processing device comprising:
an impedance calculator for computing impedance information of each of the test parts based on the digital response signal corresponding thereto;
an image generator operable to generate an image signal according to the impedance information from said impedance calculator and coordinate signals corresponding to respective locations of the test parts on the biological tissue; and
a judging unit operable to determine whether or not the biological tissue is normal according to the impedance information and the coordinate signals that correspond to the test parts of the biological tissue.

14. The data processing device as claimed in claim 13, further comprising a display for displaying an image corresponding to the image signal.

15. The data processing device as claimed in claim 13, wherein:
said image generator is configured to obtain a maximum value H and a minimum value L among the impedance information for the test parts of the biological tissue;
when a value M associated with a normal impedance range of the biological tissue is closer to the maximum value H than the minimum value L, said image generator is configured to generate grayscale values G according to G=D/[(H−M)/(0.5R−1)], wherein D represents the impedance information, and R represents a total number of grayscale levels; and
when the value M associated with the normal impedance range of the biological tissue is closer to the minimum value L than the maximum value H, said image generator is configured to generate the grayscale values G according to G=D/[(M−L)/(0.5R−1)].

16. The data processing device as claimed in claim 15, wherein the value M associated with the normal impedance range of the biological tissue is an average of upper and lower limits of the normal impedance range of the biological tissue.

17. The data processing device as claimed in claim 15, wherein said image generator is configured to generate the image signal according to the grayscale values G and the coordinate signals that correspond to the test parts of the biological tissue.
